(19) United States
(12) Patent Application Publication
(10) Pub. No.: US 2022/0172807 A1
(43) Pub. Date: Jun. 2, 2022

(54) COMPUTING METHOD AND SYSTEM FOR COMPUTING A MEDICAL CARE QUALITY ESTIMATION VALUE

(71) Applicant: Watershed Health LLC, Daphne, AL (US)

(72) Inventor: Arthur G. Grant, III, New Orleans, LA (US)

(21) Appl. No.: 17/673,927

(22) Filed: Feb. 17, 2022

Related U.S. Application Data
(63) Continuation-in-part of application No. 16/508,184, filed on Jul. 10, 2019.

(51) Int. Cl. G16H 10/60; G16H 10/20; G16H 50/20; G16H 40/20

(52) U.S. Cl. CPC G16H 10/60; G16H 40/20; G16H 50/20; G16H 10/20

(57) ABSTRACT

The present invention provides for computing a quality factor value associated with medical care by receiving data sets associated with medical care and analyzing data of the data sets using a medical event detection routine to determine data relationships. The method and system detects a medical event and a care provider based at least on the plurality of relationships. The method and system detects a second event and a first number of data points in relation to the medical event. The method and system also detects a second number of data points relating to a completed standard-of-care response to the second event. The method and system determines a response timeliness value and a response completeness value based on the data analysis. The quality factor value is calculated based on the response completeness value and the response timeliness value.

### RELATED APPLICATIONS

The present application is a continuation-in-part of, and claims priority to, U.S. patent application Ser. No. 16/508,184 entitled “METHOD AND SYSTEM FOR ESTIMATING HEALTHCARE SERVICE QUALITY” filed Jul. 10, 2019, the entirety of which is incorporated herein.

### COPYRIGHT NOTICE

A portion of the disclosure of this patent document contains material that is subject to copyright protection. The copyright owner has no objection to the facsimile reproduction by anyone of the patent document or the patent disclosure as it appears in the Patent and Trademark Office patent files or records, but otherwise reserves all copyright rights whatsoever.

### FIELD OF THE INVENTION

The present invention relates generally to multi-database data analysis and generating a computational value output and more specifically to electronically computing a quality factor value associated with medical care quality factor value based on machine learning analysis of data sets.

### BACKGROUND

There is significant growth in the amount of healthcare related electronic data, such as generated through electronic medical records, electronic devices, and other sources. While the volume of data continues to grow, problems remain in translating this data into actionable results for providing optimal patient care and motivating healthcare providers to provide the highest quality of patient care.

Many of the existing computer processing techniques for improving patient care revolve around analyzing data, but fail to generate actionable results accounting for quality assurance. For example, current techniques seek to create electronic exchanges between various providers within the healthcare continuum, e.g. hospital and physician practices. But these current techniques omit a large portion of care providers, such as skilled nursing facilities, physical therapists, occupational therapists, pharmacies, home healthcare agencies, hospice, emergency medical services, etc.

In recent years, quality data has become available from the Centers for Medicare and Medicaid (“CMS Data”). Studies have determined that CMS Data operates in a purely backwards looking manner and there is poor accuracy for determining future patient quality care based solely on the CMS Data.

One current proposed solution for improving healthcare is described in U.S. Publication No. 2018/0182475 (“475 Publication”), including monitoring various data feeds and using artificial intelligence (“AI”) to detect a significant event or condition. Upon detection, the '475 Publication can then estimate likely next steps and/or generate a course of treatment. This solution is static based on pre-existing medical guidelines, but does not account for patient quality of care or generate a form of quality assurance for the patient.

Another solution is described in U.S. Pat. No. 8,069,080 (“'080 patent), generating healthcare provider quality and cost ratings. Where the '080 patent generates provider quality data, this provider quality is based on the post-care analysis of treatment data. The '080 patent does not account for any real-time or just-in-time post-care or incident inquiries from healthcare providers. Moreover, the '080 patent analysis is independent of any particular patient.

There are techniques for using a wide variety of data sources to generate better or improved data analysis, such as U.S. Publication No. 2018/0121857 (“'857 Publication”). The '857 Publication describes managing healthcare data in a healthcare operating system, the system having multiple individual systems or processing modules. The '857 Publication provides for managing these large networks of data, but like other solutions, does not and cannot provide quality assurance or engender quality of care based decision making, lacking real time or just-in-time data analytics.

Thus, while techniques exist for managing healthcare data from multiple sources, none of these systems can account for or provide quality assurance measures, including motivating healthcare providers to improve quality of care.

### BRIEF DESCRIPTION

The present invention provides a computerized method and processing system for computing a quality factor value associated with medical care.

The method and system operates in a networked processing environment, for example across an Internet or private-network. The method and system includes receiving a plurality of data sets associated with medical care. The data sets can be received from a number of different data sources, databases. The databases can have medical event data stored therein or in other embodiments can be non-medical event data.

A computing engine analyzes the data of the data sets using a medical event detection routine. The routine is a processing operation to determine relationships between the data. Based thereon, the method and system detects a medical event and a care provider associated with the medical event. For example, a medical event can be a hospital discharge and the care provider can be the at-home service provider assigned to the patient. Another example of a medical event may be a standard of care practice, such as regular data entry of vital statistics for a hospitalized patient and the care provider can be the hospital.

The method and system further analyzes the data from the data sets to detect a second event related to the medical event. The second event can be any suitable type of event that occurs or is related to the medical event. For example, if the medical event is the hospital discharge, the second event can be the aftercare protocols performed by the at-home service provider assigned to the patient. In the example of the medical event being standard of care practices of regular data entry of vital statistics, the second event is the actual data entry records for the patient entered into the medical records system by the hospital care provider.

Based on analyzing the data of the data sets, the method and system further determines a first number of data points associated with the second event. The first number of data points represent the number of data elements, e.g. data input fields or recorded actions, performed by or associated with the care provider in relation to the medical event. In the example of the hospital discharge being the medical event, the first number of data points can be the recorded activities and data input related to the aftercare protocols performed by the at-home service provider assigned to the patient. In the example of vital statistics, the first number of data points can be the vital statistic information entered by the hospital care provider into the medical records system.

The data analysis further determines a second number of data points associated with the second event. The second number of data points represent the number of data elements, e.g. data input fields, associated with a proper standard of care associated with second event. In the example of the hospital discharge medical event and the second event is aftercare, the second number of data points can be the requisite number of data entry records to be submitted by the after-care provider. In the example of vital statistics, the second number of data points can be the number of statistics to be entered, e.g. blood pressure, blood oxygen readings, medications, patient responsiveness, pain levels, etc.

The method and system therein electronically determines a response timeliness value and a response completeness value using the data analysis.

The response timeliness value indicates a difference between the medical event and the second event performed by the care provider. In the example of hospital discharge, the time difference can be the difference between when the after-care provider was supposed to do things and when things were actually done, e.g. when a home visit was to be conducted and when it was actually conducted, or in another example the time difference can be when the event occurred and when it was actually documented in the medical records system.

The response completeness value is based on a comparison of the first number of data points with the second number of data points. This comparison indicates a completeness level by the care provider. In the hospital discharge example, a completeness value for timely and regular home visits to the patient can be determined by comparing the first number of actual home visits with the second number of standard home visits. In the example of vital statistics, a completeness value for regular data entry of vital statistics can be determined by comparing the first number of actual vital statistics entered with the second number of standard vital statistics entered.

Therein, the method and system calculates a quality factor value for the care provider based on the response timeliness value and the response completeness value.

In one embodiment, an electronic survey for the care provider can be the second event, the first number being a number of survey questions and the second number being a number of answered questions. The method and system therein generates an electronic survey having multiple survey questions based on the medical event. Based on determining the care provider associated with the medical event, the method and system includes electronically distributing the electronic survey to the care provider, distribution being across the network connection.

At some point in the future, a user can electronically log into a connected computing device and take the electronic survey by electronically submitting answers via an electronic user interface. The method and system includes receiving the survey response via the networked connection, the survey response including a plurality of answers to the survey questions.

The method and system therein electronically analyzes the survey response, including calculating a response timeliness value associated with the survey response and calculating a response completeness value associated with the survey response.

The response timeliness value indicates a time between when the electronic survey was distributed to the recipient and when the response was received. The response completeness value indicates how completely the survey questions were answered in the survey response, wherein the answering of the questions is independent of an accuracy or correctness of a submitted answer.

In a further embodiment, the method and system includes storing the quality factor value within profile data of the medical care provider. In one embodiment, this value can be stored in a medical care database.

In one embodiment, the method and system may receive a medical care inquiry, such as a regarding a patient. For example, the medical care inquiry may be a recommendation request for an in-home care provider. Based on this medical care inquiry, the method and system further accesses the medical care database having profile data for medical care providers and generates a provider list. The list may then be supplemented based on the quality factor value(s). The method and system may further include electronically generating a medical care provider recommendation based on the updating of the provider list.

The survey questions within the electronic survey can provide for a root cause analysis of the medical anomaly event. Moreover, the medical anomaly event can be a negative event relating to medical care of a patient. For example, a negative event can be a patient being re-admitted to the hospital within thirty days of discharge.

In another embodiment, the medical event may be a normal or non-negative event relating to the patient. Where a negative event relates to a negative outcome or scare for the patient, a normal event can be a standard event. For example, a normal event can be a timely measurement of a patient's vitals during a hospital stay. By contrast a negative event can be an adverse outcome for the patient because the vitals were not read or were misread.

The medical event detection routine may additionally incorporate or utilize machine learning operations. The plurality of data sets are not necessarily related, thus the medical event detection routine can uses machine learning operations to not only detect the relationships but better recognize interrelation between various data points.

Therein, the present method and system uses computational analysis of data sets to determine an event occurrence. Using this analysis, the method and system electronically computes the quality factor value attributable to the care provider. The full data analysis can be performed on existing data sets or in another embodiment can include interaction with users/care providers to supplement the analysis.

### BRIEF DESCRIPTION OF THE DRAWINGS

FIG. 1 illustrates a block diagram of a processing system for generating healthcare recommendations;

FIG. 2 illustrates a block diagram of the patient-centered healthcare network including data collection and exchange operations;

FIG. 3 illustrates one embodiment of the processing system of FIG. 1;

FIG. 4a illustrates a flowchart of steps of a method for estimating healthcare service quality;

FIG. 4b illustrates a flowchart of further steps of the method for estimating healthcare service quality;

FIG. 5 illustrates a flowchart of the steps a method for estimating healthcare service quality in response to a medical incident;

FIG. 6 illustrates a flowchart of the steps of a method for estimating healthcare service quality based on a communications between service providers;

FIG. 7 illustrates a screenshot of engagements with a service provider;

FIG. 8 illustrates a screenshot of generating a medical inquiry;

FIG. 9 illustrates a sample screenshot of a provider recommendation;

FIG. 10 illustrates one embodiment of a processing system for generating a quality factor value;

FIG. 11 illustrates one embodiment of the processing device of FIG. 10; and

FIG. 12 illustrates one embodiment of a data processing methodology for generating a quality factor value.

A better understanding of the disclosed technology will be obtained from the following detailed description of the preferred embodiments taken in conjunction with the drawings and the attached claims.

### DETAILED DESCRIPTION

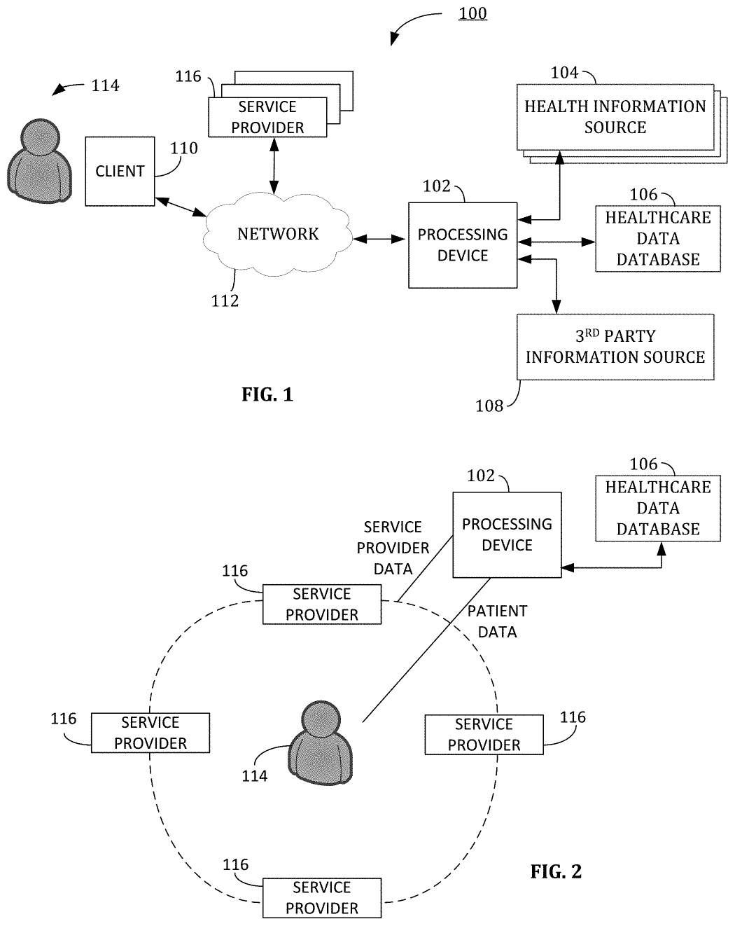

FIG. 1 illustrates a block diagram of a system 100 for facilitating data exchange, enabling multi-party communication and estimating healthcare service quality for patient. A patient may be any person or persons requiring or seeking medical care information. The term patient may specifically refer to the person needing the medical assistance, but can also refer generally to a person and family, care providers, or any other assistant or support structure relating to medical care.

The processing system 100 of FIG. 1 illustrates a processing device 102 accessing external data sources. In the embodiment of FIG. 1, the sources include health information sources 104, healthcare network database 106, and third party information sources 108. These sources may be locally stored and connected, but also may be network connected, such as located in a cloud-based computing or networking system. Additionally, these sources can be public or proprietary data sources.

The processing system 100 operates a software platform enabling service providers to easily practice three important behaviors in clinical care with other members on the network: communication; data exchange; and quality assurance. The system 100 measures the behaviors of the healthcare providers and therein estimates quality assurances of service based on behavior metrics. The system 100 allows for patients and providers to electronically build patient-centered healthcare networks using real-time metrics to guide decision making.

In the system 100, the processing device 102 engages a client device 110 via the network 112, providing the interface for a user 114. The processing device further engages service providers 116. Service providers 116 may be any individual or organization providing healthcare or related services, including for example, but not limited to, hospitals, pharmacies, emergency medical services, residential care facilities, individual doctors/nurses.

While not illustrated, the service providers 116 further include user interface functionality for communicating with the processing device 102. Communications with the processing device 102 can include sharing operational data, interacting with patient records, submitting patient information, communicating with other service providers or patients via the platform, by way of example.

The processing device 102 may be any suitable device or devices for performing processing operations as described herein. The processing device 102 may be a unitary processing device or system or can be a distributed processing system, such as one or more processing networks disposed in a networked or cloud based computing platform. As described in further detail herein, the operations of the processing device are performed in response to executable instructions from one or more computer readable medium, such as a memory device having the instructions stored therein.

The health information source 104 can be any suitable source for health information or health related data, such as locally or networked sources, including public and private data sources. By way of example, health information source 104 can be electronic medical records. In another example, the source 104 can be a medical billing and/or insurance data. In another example, the health information source can include CMS Data or similarly generated qualitative data.

Another sample of a source 104 can be information about medical doctors or medical facilities, such as assembled by external or monitoring sources. For example, for medical facilities may track patient care data such as average length of stay for specific procedures, re-admission rates, patient demographic data, etc.

The healthcare network database 106 includes stored data generated within the patient-centered healthcare network via the processing device 102. As service providers 116 engage the processing device 102, they submit data and those interactions are then stored within the database 106. For example, service provider 116 can enter operational data relating to day to day operations, including interactions with patients.

Similarly, as users interact with the processing device 102, such as submission of health data or other interactions, that information is additionally stored in the database 106.

Third party information source 108 can be any other source not categorized as a health information source 104. The third party information source 108 can be any other suitable information usable for assisting in or being a computational factor in the processing operations. By way of example, one source 108 may be map data for providing distances, such as an estimated distance between a residential care facility and the patient's home address (e.g. family). In another example, one source 108 can be weather information predicting bad weather (e.g. snowstorm, hurricane, etc.).

The client 110 may be any suitable processing device or devices operative for engaging the user 114. The client 110 may include dedicated or installed executable software for local execution, such as a local downloadable application. The client 110 may utilize any suitable browser application and run the browser as the processing gateway for network-based functionality. The client 110 can be a stationary device, such as a desktop computer, or can be a mobile computing device having interconnectivity to the processing device 102.

The network 112 can be any suitable public or private network, or a combination thereof. In one embodiment, the network 112 is the Internet, but may also include local area networks and/or mobile networks facilitating engagement and communication thereacross.

The user 114 can be one or more users engaging the client device 110 for seeking a healthcare recommendation. For example, the user 114 can be a hospital administrator working with the patient for providing the recommendation or referral. Similarly, the user 114 can be the patient, a guardian, caretaker, etc. The user 114 may also be a service provider seeking a recommendation or referral for another service provider. As used herein, any referral or related output can include necessary statement or precautions ensuring compliance with regulatory and anti-kickback regulations.

Within FIG. 1, the service provider 116 can also be a person, facility, organization, company, producer, insurer, or other entity that provides medical or medical-related services. The service providers 116 interacts with the processing device 102 via the network 112.

As illustrated in FIG. 1, the processing device 102 engages any number of health information sources 104, the database 106, third party information sources 108, as well as communicating with any number of service providers 116.

The system 100 uses the processing device 102 to estimate the healthcare service quality based on service provider 116 behavior metrics.

FIG. 2 illustrates a block diagram of the patient-centered healthcare network. The processing device 102 runs the communication platform engaging with users 104 and service providers 116, storing data in the database 106.

The service providers 116 run and manage operations using the communication platform. By way of example, a service provider can manage a patient visit, such as scheduling the visit, accessing medical records, processing patient intake, processing billing, recording and updating medical records from visit, and patient follow-up post-visit.

As the service provider uses the processing device 102, the exchange of data is stored in the database 106. Data can be relative to patients, but data can also be service provider day-to-day operations. Data stored in the database 106 can include, but not limited to, clinical behavior data, communication behavior, data sharing behavior, and quality assurance behavior.

Similarly, the database 106 can store patient data. Patient data may include, for example, demographic information, hospitalization information (admission, discharge, transfer information), medical and related documents, medication information, vital sign information, and test result data. In some embodiments, the patient may submit this information directly, such as submitting documents and filling out a medical background survey, intake form. In another embodiment, the data may be collected from other sources, such as wirelessly connected devices for example a pedometer measuring exercise, a heart rate monitor, etc.

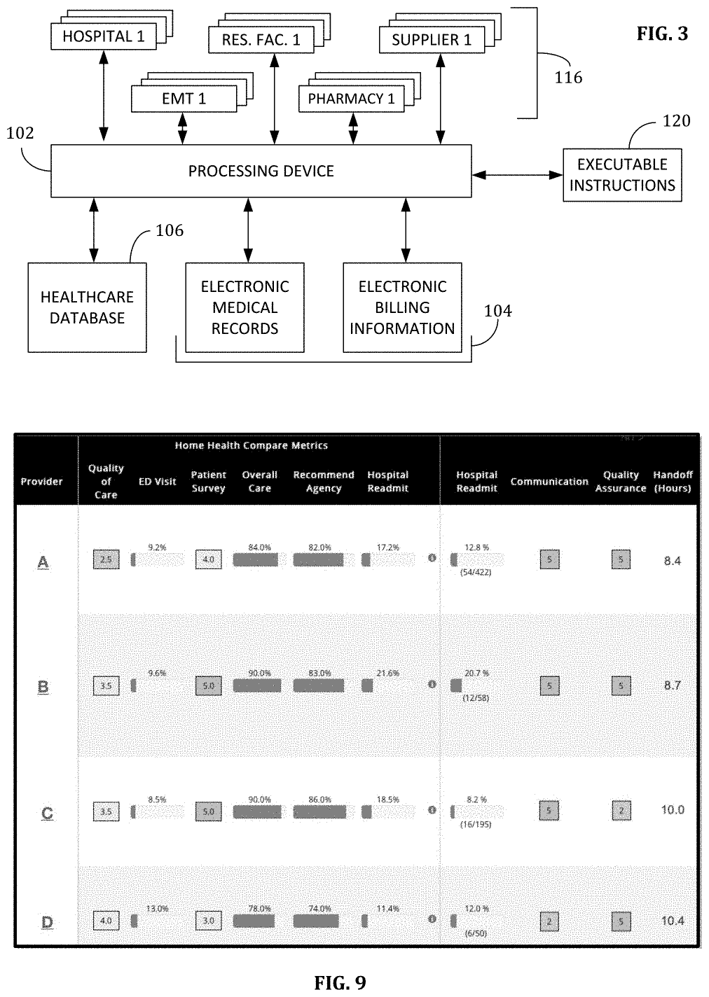

FIG. 3 illustrates a further embodiment of part of the system 100 of FIG. 1. The processing device 102 engages a plurality of service providers 116. The processing device 102 receives executable instructions stored in a computer readable medium 120.

The processing device 102 can include user and service provider registrations, as well as HIPPA compliance, data encryption and secure handling, as well as other recognized system safeguards.

FIG. 3 illustrates numerous exemplary service providers 116 engaging the processing device 102. The listed examples of service providers include various hospitals, emergency medical transport services, residential care facilities, pharmacies, and suppliers. These are exemplary providers and FIG. 3 is not a restrictive or exhaustive list.

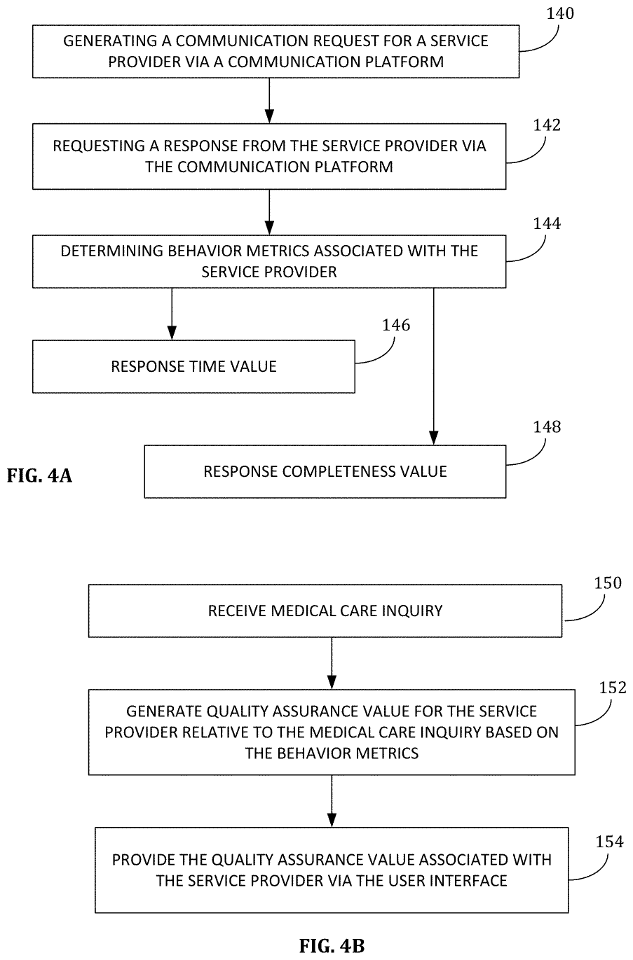

FIG. 4a illustrates a flowchart of a method for estimating healthcare service quality, using the system 100 of FIG. 1. Step 140 is generating a communication request for a service provider via the communication platform. The request can be any suitable type of request, including request for communication, data exchange, and provider behavior.

Step 142 is requesting a response from the service provider to the communication request. In one embodiment, the request of a response may be based on a notification requesting a response. In another embodiment, the request of the response can be based on predetermined operations by the service provider, for example when seeing a patient, the service provider should then enter patient medical information.

Step 144 is determining behavior metrics associated with the service provider. Determining these behavior metrics is based on tracking interactions by the service provider with the communication platform.

The first behavior metric is determining a response time value, step 146. The second behavior metric is determining a response quality value, step 148.

The timeliness relates to how quickly the service provider responds to the inquiry, such as tracking within the context of hours or days. The completeness of the response relates to addressing all the questions and providing complete responses.

Timeliness, as used herein, relates to the amount of time between two measurable events. For communication, timeliness can be measured as the elapsed time between submission of an inquiry to the service provider and response receipt. For data exchange, timeliness can be measured as the elapsed time between when a data request is made to the service provider and when the requested data is provided. For clinical behavior, timeliness can be measured as the elapsed time between one clinical event, such as hospital discharge, and another clinical event, such as a setting a follow-up office visit. For quality assurance, timeliness can be measured as the elapsed time between when a request to perform root-cause analysis of an event is made to the service provider and when the service provider answers questions allowing for the root-cause analysis to be performed.

Timeliness can be a defined scale as predetermined by the system or a system administrator. By way of example, a response within the first 60 minutes can receive a top score, with the score being reduced for future timed interval (e.g. 120 minutes) during normal business hours. Timeliness can also be a relative scale predicated on responsiveness of other service providers. For example, if the average response time is 6 hours, a response sooner than 6 hours can have a higher rating and a response time longer than 6 hours can have a lower rating.

Similarly, completeness can be a relative or preset scale. One technique for completeness can be indicating if an answer is received. For example, the inquiry may include yes/no questions and monitor receipt of the response. In this example, the completeness is not predicated on a type of answer, rather the actual submission of an answer, any answer. In another example, for open-ended questions, completeness may include any other suitable value such as counting the number of words of an answer, where a three word answer can indicate an incomplete response but a twenty-five word answer indicates a completed response.

FIG. 4a relates to collecting these behavior metrics. By way of example, the steps of FIG. 4a can be repeated for each communication request, the behavior metrics being collected over a period of time.

By contrast, FIG. 4b illustrates steps using the collected behavior metrics for estimating healthcare service quality. Step 150 is receiving a medical care inquiry, relating to a patient or a user request. For example as illustrated in FIG. 1, the user 114 may access the processing device 102 via the client device 110, requesting a medical service. The medical care inquiry, being related to a patient, can also be submitted by a service provider, for example a service provider seeking another service provider to provide additional resources or services to the patient.

For example, the medical care inquiry can be to request medical discharge from a hospital and placement in a residential facility. This inquiry can include one or more service providers, e.g. medical transport services, residential facility services, pharmacy services, physical therapy services, etc.

With these services, there are associated attributes. These attributes can be any suitable attributes as recognized by one skilled in the art. The attributes can relate to quality of care and patient safety. In the above example of medical transport services, attributes can include availability of vehicles, driver safety record, etc. In the above example of residential facility services, the attributes can include nurse-to-patient ratios, hospital re-admission rate, distance to emergency room, etc.

Step 152 is generating a quality assurance value for the service provider relative to the medical care inquiry, which can be based on the behavior metrics. The responsiveness of the service provider to response requests from the communication platform indicates higher quality of service provider attention to detail. Therefore, these behavior metrics are used to generate the quality assurance values.

In one example, the medical care quality value can be based on the comparison or scaling of the service providers values relative to similarly-situated service providers. In another example, the medical care quality value can be a cumulative value for the service provider based on the service provider interactions as measured in the patient-centered healthcare network.

It may be presumed that if a service provider has a high rating relative to its responsiveness to the response request, this indicates a high level of overall quality. Therefore, where the user or patient may be seeking quality assurance for a service provider providing service A, e.g. timeliness of medical transport, the provider's high ranking can indicate reliability in service B, e.g. having readily-available medical services during transport.

Step 154 is providing the quality assurance value associated with the service provider, estimating healthcare service quality for the patient. This value is presented via a user interface accessing the communication platform, such as via a user interface running on the client device 110 accessing the processing device 102 via the network 112 of FIG. 1. In another example, the quality assurance value can be provided to another service provider via the user interface.

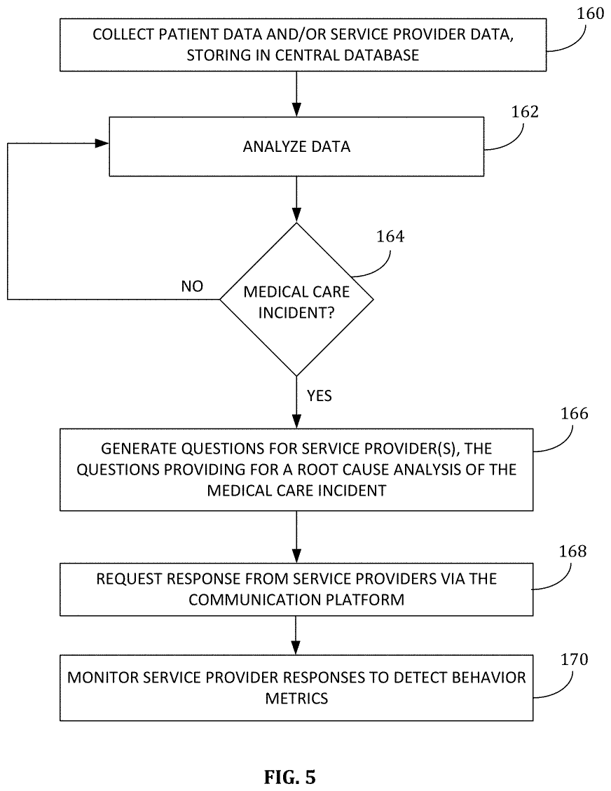

FIG. 5 illustrates a flowchart of steps of one embodiment of estimating healthcare service quality based on communication based on a medical care incident. Step 160 is collecting patient data and service provider data, as well as storing said data in a central database. As noted above, the patient data can be any suitable data relating to the patient and the service provider data can include operational data, as well as any other related data.

Step 162 is to analyze the data. Analysis includes step 164, determining if a medical care incident occurs. A medical care incident can be an anomalous event, for example a negative event. The event can occur with a patient or can be more general. In one example, an event can be a patient being re-admitted to a hospital within thirty days of hospital discharge. Monitoring patient data can detect a hospital discharge and a hospital re-admission occurring within the defined time period. Moreover, an anomalous event does not expressly indicate or imply a negative occurrence, but can include general standard of care inquires.

In another embodiment, a medical care incident may be based on user or service provider input. For example, an administrator may manually enter an event or a doctor may request investigation to assist in further medical care for the patient.

If detected, step 166 is to generate questions for service provider(s) relating to the medical care incident. In one embodiment, the questions may be directed for providing a root cause analysis of the incident

In one embodiment, the method may determine all connected service providers associated with the event. This determination may be performed using the patient data as well as other data. Similarly, the determination may be performed by an administrator or other user manually selecting or designating service providers.

Maintaining the above example of hospital re-admission within a preset time from discharge, the questions may be standard questions relating to the patient's health and monitoring of health prior to discharge. The service provider here may be the hospital facility, nurses, and doctors associated with the patient. Another service provider may be the pharmacy if prescriptions were issued.

In one embodiment, the system may include pre-generated questions relating to an incident. Questions can include standard questions relating to the service providers role in the incident. In another embodiment, an external engine such as an artificial intelligence engine, can generate specific questions for the various service providers.

Step 168 is to request a response from the service provides via the communication platform, similar to step 142 of FIG. 4 above. Step 170 is monitoring the service providers responses to detect behavior metrics, similar to steps 144-148 of FIG. 4 above.

Therein, the method monitors how service providers respond to requests relating to medical care incidents. The timeliness and completeness of the response again translates to assurance of quality, e.g. attention to detail, by the service provider. Thus, this method provides additional metrics for generating quality assurance values for service providers.

Another embodiment is monitoring operational data for detecting behavior metrics associated with the standard data entry. Under normal operations, the service providers should be entering this operational data in a regular and timely manner. This regular and timely (and complete) data entry indicates a higher degree of quality of care by the service provider.

As used herein, operational data can relate to operational features of the service provider engaging in its normal practices. Operational features can include the elapsed time between two events (e.g., hospital discharge and a post-discharge office visit) when that information is either manually entered into the system or electronically fed into the system via some type of electronic interface. It can also include the completeness of doing some measurable task (e.g., administering an insulin injection every day) when that information is either manually entered into the system or electronically fed into the system via some type of electronic interface. The platform, colleting this operational data, therein monitors the timeliness and completeness of these activities by the service providers. This data is already being collected, which now is usable for estimating quality assurance.

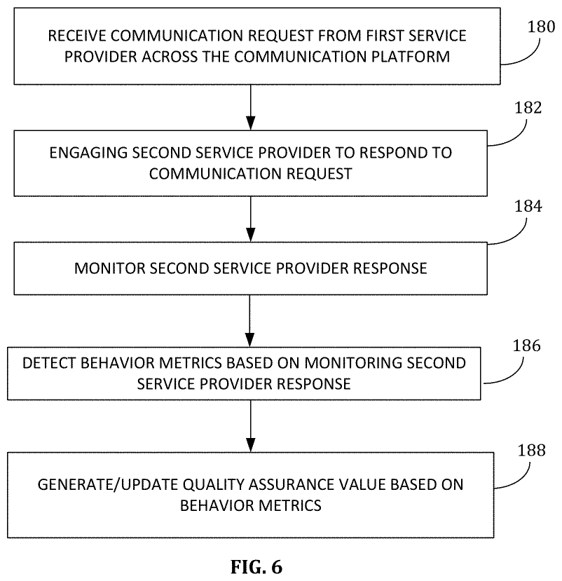

FIG. 6 illustrates another manner of service provider behavior, responding to communication requests across the communication platform. Service providers can communicate across the platform, including requesting information and/or data from other service providers. For example, a first service provider can request patient information or submit questions, for example requesting or asking about a patient's prescribed medicines from a second service provider. In another example, the first service provider might request patient records, x-ray image files, or other type of data. How the second service provider responds to those requests can indicate quality assurance.

Step 180 is to receive a communication request from a first service provider. One example can be a patient having a consultation with the first service provider, and the patient's regular doctor is the second service provider. The first service provider can request information on the current medications, including timing and dosage. This request is processed across the communication platform.

Step 182 is engaging the second service provider to respond to the communication request. Step 184 is then monitoring the second service provider response, including if/when the service provider responds to the inquiry.

Step 186 is detecting the behavior metrics therefrom. For example, if the second service provider quickly and completely answers the inquiry, this can indicate a high quality assurance value. Similarly, if the second service provider quickly responds, but gives incomplete information, e.g. provides prescription dosage information but omits timing information, or fails to timely respond, this can indicate a lower quality assurance value.

Step 188 is to generate and/or update quality assurance values based on the detected behavior metrics. Step 188 can therein provide information usable for presentation to a patient, user, or other system operator seeking quality assurance information.

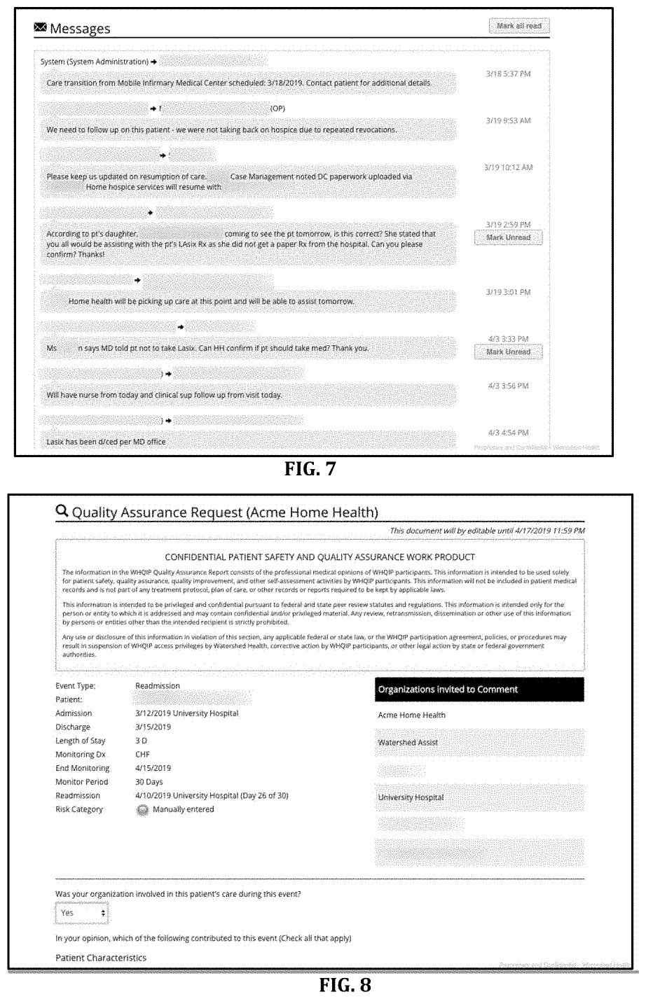

For further examples of the user interface and service provider interactions, FIG. 7 is a sample screenshot of an inquiry to the service provider via the user interface. Similarly, FIG. 8 is a sample screenshot of a user interface screen for generating a medical care inquiry.

FIG. 9 is a sample screenshot of an output display including the ranking of service providers. Each service provider (A-D) has various metrics with associated values. The ranking of the providers generates the recommendation. In this sample screenshot, the system metrics including both a quality assurance metric and a communication metric. Where provider A and provider B has identical quality assurance values (“5”), provider A incudes a higher communication factor and is thus recommended higher within the display rankings.

In another embodiment, the method and system may additionally use the third party information sources for assisting in recommendations. As described above, these sources can be any information usable for facilitating an informed decision by the patient or administrator. One example noted above is map data including mileage. The method and system can then update or modify recommendations based on this map data. For example using the FIG. 7 screenshot, if Provider A was located 25 miles from the patient's family and Provider B was located 2.5 miles, the inclusion of mileage may change the listed order of providers. In this case, the ease of family access can directly relate to the ability for the patient to be visited by family, improving overall mood for the patient and can thus increase quality of medical care.

In one embodiment, the healthcare management system described herein can include user interface functionality for the user to include or remove these third party factors as requested for improving search results. Again using the distance example, if the patient does not have family in the area, the distance between the facility and the patient's home can thus be ignored or its value reduced.

In another embodiment, the system may additionally use CMS Data as additional input for estimating healthcare service quality.

The above embodiments relate to a service provider seeking a referral and using the quality assurance value as part of the referral analysis. A further embodiment can include a service provider using the quality assurance value of another service provider in deciding to accept a referral.

For example, service provider A seeks to refer a new patient to a service provider B, but service provider B is unsure about accepting this new patient. Service provider B can review the quality assurance value of service provider A. In this example, if service provider A has a low quality assurance value, service provider B may not want the referral because of concerns about timely receipt of responses to communications about the patient. By contrast, if service provider A has a high quality assurance value, service provider B can accept the referral with greater confidence.

The method and system, operating within the patient-centered healthcare network, motivates service providers to provide the highest quality of services. The system and method generates ranking of service providers recommendations based on the information collected within the system.

The system monitors, records, and tracks daily on-going transactions with patients and service providers. The system records just-in-time activities, logging interactions into the central platform.

When an event occurs, the system can detect the event and inquire with various service providers as to determine potential causation. The method and system can therein provide root cause analysis of why things go wrong. Part of that analysis includes providers providing complete and timely responses to the inquiries.

As providers improve responsiveness to medical care inquiries, the providers quality assurance value increase. The service provider being responsive to the inquiries is a key indicator of service quality and usable for generating the medical care quality value and subsequent rankings.

The method and system estimates healthcare service quality by monitoring the service providers. Therein, the system and method motivates service providers to provide not only the highest quality of care, but be additionally responsive to the inquiries to improve quality rankings within the patient-centered healthcare network.

The method and system provides a software platform that facilitates inter-provider communication (including root-cause analysis) and data exchange. The method and system quality assurance because increased communication and data exchange leads to better service provider quality.

In the method and system, service providers can use these behavior metrics to inform choices about building patient-centric care networks. Making informed decisions based on quality assurance values associated with service providers will be, by definition, make better networks for the user/patient.

The system and method is additionally usable by service providers. They can gain insight about their own strengths and weaknesses, such as determining or viewing their own quality assurance values. The service providers can use these quality assurance values to inform business decisions such as whether to enter a risk-based contract with another provider. Similarly the method and system can be used to inform patient-level decisions such as choosing the best performing provider (e.g., best nursing home) when building a patient-centered care network.

The method and system further enables the referral of patients from one provider to another using the system. The system-generated metrics are seamlessly integrated into this referral process by presenting metric information to system users during the provider selection process. The referral process can be predicated on assurance values, as well as other data, operating in compliance with all required laws, including anti-kickback laws.

The method and system therein improves patient care. Service providers have a natural incentive to obtain better metrics in order to receive more referrals. Within this system, better metrics are obtained by optimizing communication, data exchange as well as certain measured provider behaviors.

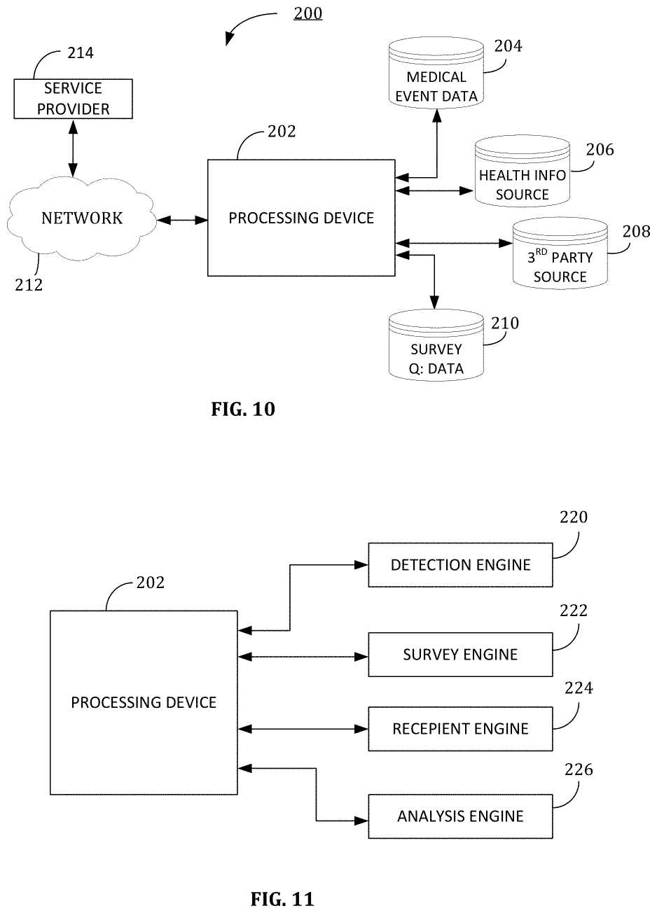

FIG. 10 illustrates one embodiment of a computerizing processing system providing for generating a quality factor value. The system 200 include a processing device 202 in communication with multiple databases, including a medical event database 204, a health information source data database 206, a third party source database 208, and a survey question data database 210. The processing device is connected to a network 212 and communicates with a service provider 214 thereacross.

The processing device 202 may be one or more processing devices disposed in a networked or computing environment. In one example, the processing device 202 operates in a cloud-based processing environment. In another embodiment, the processing device 202 can be a dedicated server or servers performing the processing operations as described herein. The processing device performs the processing operations in response to executable instructions stored in a non-transitory computer readable medium, as recognized by one skilled in the art.

The medical event data database 204 stores medical event data. The medical event data can be any form of electronic medical records, including for example medical records submitted by practitioners on an on-going basis. The medical event data database 204 may include directly-entered data or can include modified or translated data from varying medical record systems.

For example, medical care providers can enter data into an Epic System® software application. This data recording on-going medical care parameters, such as when patients are admitted, administering medications, hourly or periodic status reports, or any other suitable electronic medical record data. In one embodiment, a translation application may be utilized for translating or reformatting medical event data into a format usable by the processing device 202.

The health information source database 206 stores standard or normative care values. This data is usable, as noted below, for detecting when medical event data is outside a normal range and triggers care inquiries. One example can be a standard indicating that a properly attended patient should not be re-admitted for medical care within at least 30 days of discharge. Another example can be detecting an emergency-care event, such as an adverse reaction to a medication, a recurring medical condition, or other events.

In one embodiment, the normative care values can additionally include routine practice events and detecting or establishing medical care inquiries based on routine events. Herein, the determining of a medical anomaly event is not restricted to a negative event associated with patient care, but can also be based on medical care provider behaviors. For example, one type of event can be entry of patient records.

The third party source database 208 is data acquired from one or more third party sources. One example of this source data can include Medicare CMS data. This database 208 includes any usable data acquired or sourced external to the medical event data 204. In further embodiments, the database 208 may not be related to medical care, but can be any suitable type of data. For example, the database can be a dietary tracking app database, a movement or calorie tracking app database, credit card or financial data, or other types of data by way of example.

The survey question data database 210 is a database or dataset including survey question data. These can include standard questions to be included in a survey. These also can include unique or specific questions based on the medical event data and/or the anomaly event. For example, basic questions can include request date, time of day, location, etc. Specific questions are context-specific, for example if the anomaly is an adverse drug reaction, a survey can include a question asking if the patient was asked about taking additional medications and another question is asking when the medical care provider cross-referenced a drug interaction reference guideline. Another example may be an untimely hospital re-admission, with questions relating to original discharge instructions and after care instructions, as well as contacts with after care providers.

The network 212 may be any suitable network, including a publicly available network, e.g. the Internet, and/or a private or internal network. The service provider 214 is an end user accessing server-based functionalities noted by the processing device 202. The service provider 214 may be similar or identical to the service provider 116 of FIG. 1 above.

The processing system of FIG. 10 may operate similar to the system of FIGS. 1-9 above. The generation of the quality factor value as described below can be similar to the quality assurance value. The operational factors of the FIGS. 1-9 system may use varying terminology, but include similar computational results associated with seeking feedback from medical providers and translating the feedback to estimation of care quality, including for recommendation or ranking purposes, in one embodiment.

Where FIG. 10 illustrates one embodiment of the computing system, FIG. 11 is an expanded view of the processing device 202. The device 202, in performing varying processing operations, executes a plurality of software engines. These engines may be software-based engines executing within the device 202, or can be networked or connected engines executing in other processing environments, for example in a cloud-based processing environment.

In this embodiment, the device 202 includes four exemplary engines. These engines include a detection engine 220, a survey engine 222, recipient engine 224, and an analysis engine 226. These exemplary engines are not limiting in nature, whereby the processing device 202 may execute additional engines as part of the processing operations noted herein.

The detection engine 220 performs processing operations to detect relationships between various data sets, executing the medical event detection routine as described herein. As described in greater detail below, the detection engine 220 may utilize machine learning operations to detect relationships in disparate data sets.

The survey engine 222 performs processing operations to generate one or more surveys relating to the detected event. This engine 222 can use or reference data from databases 204, 210 of FIG. 10.

The recipient engine 224 performs processing operations to determining survey recipients. This engine 224 can use or reference data from the databases 204, 206, and/or 208 of FIG. 10.

The analysis engine 226 performs processing operations to analyze the survey response. As noted below, the analysis engine calculates the response timeliness value and the response completeness value.

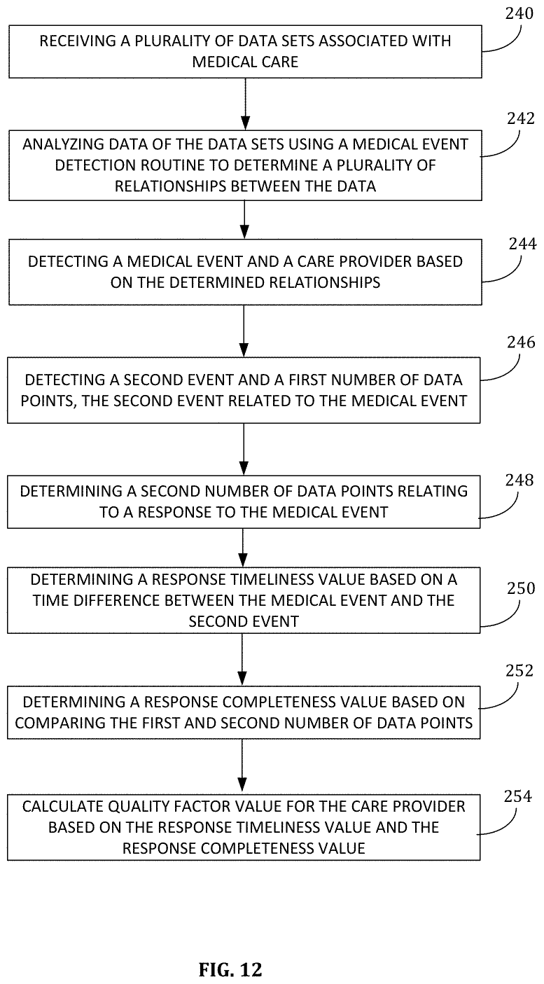

FIG. 12 illustrates a flowchart of the computing method for computing a quality factor value associated with medical care. The present computing method and system uses processing routines and may utilize machine learning operations to detect events and improve patient care. A quality factor value, as used herein, is a computational value usable for estimating a reliability or care quality associated with a care provider, the quality factor value is predicated not on the correctness of a survey response, but rather the timeliness and completeness of the survey response.

The quality factor value can be used to generate a recommendation or generated a ranked list of providers. The value can be used in conjunction with other criteria for reviewing or quantifying medical care providers.

The computerized method can include processing operations performed using the engines 220-226 and processing device 202 of FIG. 10-11, as well as accessing content data, for example in database 204-210 of FIG. 10.

Step 240 is receiving a plurality of data sets associated with medical care, the data sets received from a plurality of data sources. This can include one or more data call operations performed by the processing device 202. These data call operations can be at regular intervals, such as daily analysis or review. The data call operations can also be in response to a triggering event or notification for accessing the medical event data. For example, a triggering event can be a notification that a patient has been re-admitted to the hospital. The triggering event can be generated by monitoring the medical event data or other monitoring operations as recognized by one skilled in the art. In another embodiment, the triggering event can be manually triggered, for example by an administrator seeking to conduct surveys related to one or more events. In another embodiment, the triggering event can be a time-based trigger such as a regular monitoring of data entry or patient care records, for example a weekly survey relating to general patient care.

As used herein, receiving can also mean receiving access to data from data sources. Where possible, data may be downloaded or stored in accessible database(s), but systems may additionally sequester data or preclude offloading of data sets, for example for security reasons. Therefore, receiving a plurality of data sets can also include receiving access to data sets.

Step 242 is analyzing the data of the data sets using a medical event detection routine. The routine is a processing operation detecting relationships between various data points. The medical event detection routine can use machine learning and/or iterative processing routines to determine relationships. For example, one data set may be pharmacy database listing prescriptions, dosage, and time of day to take medicines. Another data set may be a personal health device recording heart rate information. A relationship may exist between medicines and heart rate fluctuations. The relationships between data points may be generally recognized and accepted data points but may also include new or non-traditional relationships.

Machine learning operations of the event detection routine can include iterative processing of data sets, including for example linear regression, logistical regression, decision tree analysis, etc. The routine can process the disparate data sets.

In the medical space, there are vast amounts of data resources and terabytes of data readily available. The detecting engine can process, filter, and analyze these large data sets as a foundation for improving patient care. The detection of events is the first step in the process for generating computational values usable for improving patient care.

In one embodiment, the detection engine (220 of FIG. 11) compares the event data to standards or rules. Based on this comparison, the engine determines anomaly events as being events outside of the standards, rules, guidelines, etc. Where the survey is based on a regular interval, for example administration routinely monitoring staffing operations, the medical anomaly event can be more generally referring to standard care practices or for example if a survey hasn't been taken in a designated time interval. There is no express requirement that the medical anomaly event must be a negative health event but can relate to general standard-of-care procedures of healthcare maintenance and management.

Step 244 includes electronically detecting a medical event and a care provider associated with the medical event. The medical event detection is performed by a processing device executing the medical event detection routine as noted above. The medical event can be a negative event, but the medical event can also be a non-negative event, for example routine medical care such as but not limited to filling a prescription, entering patient vitals, recording lab notes, scheduling a follow-up visit or appointment, administering routine medical care, etc.

The care provider can be any suitable person or persons associated with the patient or the event, directly or indirectly. Determining which medical care providers are related to or should be related to the event can be based on acquiring provider information from the medical records. This detection can be based on referencing a list of providers associated with a patient or a particular event.

For example, if the medical event was a patient transfer to an outpatient facility, the medical event data may include information on the original transfer but fail to indicate a notation of the transportation provider. Thus, additional data sources, such as sources 206 of FIG. 10, can be accessed to acquire additional or supplemental information. In this example, insurance information may include the name of the transportation provider as well as the name of driver and assistant(s) that assisted with the transportation.

In this embodiment, step 246 is further analyzing the data in the date sets to detect a second event and a first number of data points, the second event relating to the medical event. As noted above, the medical event can be a hospital discharge and the second event can be the after care provided to the patient by an in-home care provider. Another example from the same medical event can be a pharmacist fulfilling prescriptions for the patient prior to or concurrent with the discharge. Another example from the same medical event can be a primary care provider scheduling and seeing the patient as a follow-up medical examination after discharge.

In these examples, the first number of data points are data fields indicating data entry or data elements associated with the associated second event. For example, the in-home care provider data points can include data entry noting timing of initial contact, first visit with the patient, subsequent visits, records of actions performed during each of the visits, etc. These data points are available from the data in the data sets, mined and harvested by the method and system.

Step 248 is determining a second number of data points relating to a response to the medical event. In this step, each second event and associated care provider includes a standard of care representing a complete action response in relation to the medical event. Here, the complete action response indicates the excepted or normative standard of care. For example, for an in-home care provider, the second number of data points are data fields that can indicate the standard number of visits, the standard number of recorded data entry from various visits, and other standard data associated with proper care. This second number of data points illustrates a baseline for the normative standard of care expected for a care provider providing care to a patient or otherwise engaging in medical care.

In one embodiment, the correctness of the events indicated by the first number of data points is not considered. In this embodiment, no analysis is performed if the data entry or underlying content of the data is correct, rather the analysis is if the data exists. For example of the data points are vital statistics entered by a care provider on a regular interval, this embodiment does not determine or account for the data entry to be correct. Rather, the method and system relies on the fact that the care provider actually entered data into the system independent if their data entry is correct.

In one embodiment, a standards or second number of data points database can include these data values. The database can be a standards database. In another embodiment, data analytics using machine learning operations can build the standard of care data values by mining the data from the data sets and determining standard data points by other care providers in similarly-situated care events.

Step 250 is electronically determining a response timeliness value based on a time difference between the medical event and the second event performed by the care provider. This timeliness value can be a single value associated with a single recorded act or event, for example if the second event is filling a prescription the timeliness value can be the length of time between receipt of the prescription and filling the prescription. This timeliness value can be a conglomerated value based on timeliness of multiple acts or data points. In the example of entering vital statistics, the timeliness can be the time delay or offset from multiple data entries, e.g. if data is to be entered hourly the value can indicate how often this data entry is received offset from the hourly requirement. Therefore, the timeliness value can be calculated based on analyzing the data of the data sets and determining time differences from expected or normative time entry requirements or guidelines.

Step 252 is determining a response completeness value based on comparing the first number of data points with the second number of data points. This determining step can be performed using a direct comparison of the two values. This determining step may also include intermediate processing operations, such as weighting or factoring different data points based on analytic rubrics. For example, not all data points may carry the same weight, so some data points may be given a higher weight or value as part of the analysis. The result of step 252 is a computed value, such as a numerical value representing a comparison between the data points acquired via computation analytics of the data sets.

Step 254 is calculating the quality factor value for the recipient based on the values, including the response timeliness value and the response completeness value. This quality factor value can be a computational result based on factors or weighting of the timeliness value and the response completeness value. For example, if quality care is more dependent on timeliness, this factor may be weighted higher, or vice versa. The quality factor value can be a number or other data field, for example a rating e.g. 3 out of 5, a percentage, e.g. 72%, etc.

In one embodiment, the method and system may utilize a survey as a specific second event. Therefore, instead of or complimentary to existing data in the data sets, the system can electronically generate an electronic survey having a plurality of survey questions based on the medical event. In one embodiment, the survey engine (222 of FIG. 11) retrieves the survey question data, which can include a data set of survey questions and guidelines for an appropriate survey.

The survey can be tailored to intended recipients and tailored to events relating to the anomaly event. For example, if the anomaly event is an adverse drug reaction, questions for a pharmacist may be different from questions for a physician's assistant and attending physician who wrote the prescription. For example, if the anomaly event is a time-based or regular interval survey, the questions can be tailored to events occurring since the prior survey.

In this embodiment, the survey is distributed to the recipient, which is likely a medical care provider. This distribution can be via the network 212 to the service provider 214 of FIG. 10.

The distribution can be maintained within the computing platform, for example distribution is a notification that a survey is waiting for completion on the platform. Whereby, the survey recipient can log into the platform to complete the survey.

Once the survey is complete, the system and method receives said response. This includes receipt of the submitted information. The response typically includes a plurality of answers to the survey questions.

In one embodiment, receipt of the survey can include receiving an indication that the survey has not been received. If a generally recognized time for survey submission is set, then no response constitutes a form of response. For example, if a survey is to be responded within five days, after five days the survey response being received is a null value indicating no actual response.

The method and system electronically process the response, including calculating the response timeliness value and the response completeness value. This step can be performed by the analysis engine 226 of FIG. 11.

As used herein, the response timeliness value indicates how quickly the survey recipient responds to the survey request. This time can be measured based on when the survey is transmitted and/or when the recipient became aware of the survey.

As used herein, the response completeness value indicates how many of the survey questions were addressed. The response completeness value does not examine or account for correctness of an answer. Rather, the estimation value calculated by the computer processing system and methodology does not require analysis or indication of correctness of submitted answers.

In one embodiment, computation of the estimation value can include calculations relative to known scaling or predetermined standards. For example, if an average response time is six hours between when a survey is transmitted to the recipient and a response received, this can be a baseline or center value. For example, a straight-line curve can be used to generate a response timeliness value. In one example, the value can vary from a 1 (lowest) to a 10 (highest). Responses at or near the middle value can have a 5 value, responses received under 1 hour have a 10 value and responses received after 12 hours (or a null value for no response received) can have a 1 value.

In one embodiment, computation of the response completeness value can be based on comparison of number of questions and number of answered questions. These values can also be scaled for text-based answers versus toggle or bullet (e.g. yes/no) questions. In one embodiment, an additional analysis can scan the text-based answers to indicate a viable answer is given. This viable answer differs from a correct answer, where a viable answer can be one that at least attempts to answer the question versus the user simply entering random text.

In this computational example, supposed a survey question has ten questions. The response completeness value can be scaled on the number of answered questions or can be scaled based on a percentage of answered questions. In one example, if the value has a range from 1 (lowest) to a 10 (highest), answering all questions garners a 10 value and no answers garners a 1 value.

In one embodiment, the response completeness value computation may include additional filtering or authentication routines. While the correctness of an answer is not a factor, data analysis can detect a non-answer, where a non-answer is a non-responsive answer. For example, if the question asks for a description of the patient's status and the response is a random combination of letters and/or numbers, this can be detected as a non-answer and counted the same as being incomplete.

Therein, the computing system can utilize a computation calculation to determine the estimation value using the two values. In a non-limiting embodiment, the estimation value can be the average of the response completeness value and the response timelines value.

In further embodiments, the computing system can use any number of additional factors for computing the estimation value. The estimation value is not expressly limited to or based solely on the response timeliness value and the response completeness value. For example, the estimation value can be adjusted based on prior estimation values. In another example, the severity of the anomaly event can account for weighting of the estimation value, e.g. if the anomaly was a patient death this should garner a more focused survey response and if the anomaly was a prescription took too long to be send to the pharmacist, a slower survey response may be reasonable.

In one embodiment, machine learning may be utilized in conjunction with the processing device 202 and operations noted therein. For example, machine learning can process survey question data and facilitate improvement or refinement of survey questions included in the survey. Machine learning can analysis the types of questions presented in the surveys and correlate response values. For example, it may be determined that surveys having yes/no or toggle questions as the first three questions have a higher response timeliness value or that surveys with all yes/no or toggle questions have a significantly higher response completeness value.

Another example of machine learning can apply to determining medical event anomalies as well as intended survey recipients. In one example, detecting a failure in reduction of anomalies can indicate a need to expand the scope of survey recipients. In another example, machine learning can search for pre-anomaly events that may be related to or have some type of connection to the anomaly, whereby then generating survey(s) for these pre-anomaly events.

Whereas the methodology of FIG. 12 generates the quality factor value, this value is usable for any number of additional processing operations via the computing platform. The value can be stored as part of profile data for the medical care provider. The estimation value is one of a number of factors part of the medical care provider profile. A provider management system can be integrated or complimentary to a medical record program, the system including a medical care database. This database can have the profile data stored therein.

The computing platform can provide recommendations or augment output data using the estimation value. In one embodiment, a user, such as a care provider, can seek a recommendation for patient. One example is seeking a residential care facility after the patient is discharged from a hospital.

The computing platform can receive the patient information and a medical care inquiry, here being an inquiry needing a residential care facility. The platform can generate a result list of available facilities and can include ordering or supplementing the list based on additional factors known to the system. Here, the order of facilities can be re-arranged based on the quality factor value known for each facility. This can create a ranking or a recommendation with the facility having the highest estimation value at the top. By having the highest estimation value, the supposition is that there is a greater likelihood of quality of care for the patient.

The computing system can re-order or augment the search result list. In another embodiment, the system can formally generate a recommendation to the user entering the medical care inquiry.

Herein, the present invention computes the quality factor value based not on correct survey answers, but on timeliness and completeness of a response. This value is usable for augmenting medical care provider inquiries. The value builds upon the premise that medical care providers who are quicker to actually answer a survey and who are more complete in how they answer the survey, are more attending to their patients and provide a higher degree of care.

FIGS. 1 through 12 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. Moreover, Applicant does not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

What is claimed is:

1. A method for computing a quality factor value associated with medical care, the method comprising:
receiving a plurality of data sets having data therein associated with medical care, the data sets received from a plurality of data sources;
analyzing the data of the data sets using a medical event detection routine to determine a plurality of relationships between the data;
electronically detecting both a medical event and a care provider associated with the medical event based at least on the plurality of relationships determined by the medical event detection routine;
electronically detecting, via the data of the data sets, a second event and a first number of data points, wherein the second event is in relation to the medical event and the first number of data points are data fields representing actions by the care provider in relation to the medical event;
for the second event, determining a second number of data points, the second number of data points are data fields representing a complete action response in relation to the medical event;
determining a response timeliness value indicating a time difference between the medical event and the second event performed by the care provider;
determining a response completeness value based on comparing the first number of data points with the second number of data points; and
electronically calculating the quality factor value for the care provider based at least on the response timeliness value and the response completeness value.

2. The method of claim 1, wherein the medical event detection routine includes at least one machine learning processing routine.

3. The method of claim 1, wherein the plurality of data sources include medical record data sources and the data of the data sets include medical record data relating to patient care.

4. The method of claim 3, wherein the medical event is a negative event relating to medical care of a patient.

5. The method of claim 1 further comprising:
in response to the detecting of the medical event and the care provider, generating an electronic survey having a plurality of electronic questions therein, the plurality of electronic questions being the second number of data points;
electronically transmitting the electronic survey to the care provider via a network user interface.

6. The method of claim 5 further comprising:
receiving a completed survey from the care provider, the completed survey including a plurality of answers, the plurality of answers being the first number of data points;
wherein the completed survey is the second event such that the response timeliness value indicates a time difference between the electronic transmitting of the electronic survey to the care provider and the receiving of the completed survey and the response completeness value indicates a difference between the plurality of electronic questions and the plurality of answers.

7. The method of claim 6, wherein the response completeness value is independent of a correctness for each of the plurality of answers.

8. The method of claim 1, wherein the second event is data entry of medical information and the second number of data points is the number of data entry fields for the data entry of medical information.

9. The method of claim 1 further comprising:
after electronic detection of both the medical event and the care provider associated with the medical event, generating an electronic notification to the care provider via a network user interface of the second event.

10. The method of claim 1 further comprising:
storing the quality factor value within a profile data of the care provider in a medical care database.

11. The method of claim 10 further comprising:
via a communication platform, receiving a medical care inquiry;
based on the medical care inquiry, accessing the medical care database having the profile data therein;
generating a provider list based on the profile data in relation to the medical care inquiry; and
updating the provider list based on the quality factor value associated with the care provider.

12. The method of claim 11 further comprising:
generating a care provider recommendation based on the updating of the provider list; and
electronically transmitting the care provider recommendation in response to the medical care inquiry.

13. A method for computing quality factor values associated with medical care, the method comprising:
receiving a plurality of data sets having data therein associated with medical care, the data sets received from a plurality of data sources;
analyzing the data of the data sets using a medical event detection routine to determine a plurality of relationships between the data, the medical event detection routine using at least one machine learning processing routine;
electronically detecting both a medical event and a plurality of care providers associated with the medical event based at least on the plurality of relationships determined by the medical event detection routine;
electronically detecting, via the data of the data sets, a plurality of second events, each of the second events being in relation to the medical event, for each of the following events including a first number of data points, wherein the first number of data points are data fields representing actions by each of the care provider in relation to the medical event;
for each of the second events, determining a second number of data points, the second number of data points are data fields representing a complete action response in relation to the medical event;
for each of the care providers:
determining a response timeliness value indicating a time difference between the medical event and the second event performed by the care provider;
determining a response completeness value based on comparing the first number of data points with the second number of data points; and
electronically calculating the quality factor value for the care provider based at least on the response timeliness value and the response completeness value.

14. The method of claim 13, wherein the plurality of data sources include medical record data sources and the data of the data sets include medical record data relating to patient care.

15. The method of claim 13 further comprising:
in response to the detecting of the medical event and the plurality of care providers, generating an electronic survey for each of the plurality of care providers, the electronic survey having a plurality of electronic questions therein, the plurality of electronic questions being the second number of data points;
electronically transmitting the electronic survey to each of the plurality of care provider via the network user interface;
for each of the plurality of care providers, receiving a completed survey therefrom the care provider, each of the completed surveys including a plurality of answers, the plurality of answers being the first number of data points;
wherein the completed survey is the second event such that the response timeliness value indicates a time difference between the electronic transmitting of the electronic survey to the care provider and the receiving of the completed survey and the response completeness value indicates a difference between the plurality of electronic questions and the plurality of answers.

16. The method of claim 15, wherein the response completeness value is independent of a correctness for each of the plurality of answers.

17. The method of claim 13, wherein the second event is data entry of medical information and the second number of data points is the number of data entry fields for the data entry of medical information.

18. The method of claim 13 further comprising:
for each of the plurality of care providers, storing the quality factor values within a profile data of the care provider in a medical care database.

19. The method of claim 18 further comprising:
via a communication platform, receiving a medical care inquiry;
based on the medical care inquiry, accessing the medical care database having the profile data therein;
generating a provider list based on the profile data in relation to the medical care inquiry; and
updating the provider list based on the quality factor values associated with the plurality of care providers.

20. The method of claim 19 further comprising:
generating a care provider recommendation based on the updating of the provider list; and
electronically transmitting the care provider recommendation in response to the medical care inquiry.
